(19) United States
(12) Patent Application Publication
(10) Pub. No.: US 2023/0404979 A1
(43) Pub. Date: Dec. 21, 2023

(54) ALPHA-2 ADRENERGIC RECEPTOR AGONISTS TO REDUCE MORTALITY AND IMPROVE OUTCOMES IN VIRAL RESPIRATORY SYNDROMES

(71) Applicant: RUSH UNIVERSITY MEDICAL CENTER, Chicago, IL (US)

(72) Inventors: John L. HAMILTON, Deerfield, IL (US); Markus A. WIMMER, Chicago, IL (US)

(73) Assignee: RUSH UNIVERSITY MEDICAL CENTER, Chicago, IL (US)

(21) Appl. No.: 18/033,997

(22) PCT Filed: Oct. 26, 2021
(86) PCT No.: PCT/US2021/056580

§ 371 Date: Apr. 26, 2023

Related U.S. Application Data
(60) Provisional application No. 63/105,457, filed on Oct. 26, 2020.

(51) Int. Cl. A61K 31/4168; A61K 31/517; A61K 45/06; A61P 31/14

(52) U.S. Cl. CPC A61K 31/4168; A61P 31/14; A61K 45/06; A61K 31/517

(57) ABSTRACT

Methods for treating viral respiratory syndromes are disclosed. In some aspects, the methods include administering a non-sedative therapeutically effective amount of an α-2 adrenergic receptor (AR) agonist to the subject. In some aspects, the methods include administering a therapeutically effective amount of an α-2 adrenergic receptor (AR) agonist and a therapeutically effective amount of an α-1 adrenergic receptor (AR) antagonist to the subject.

### RELATED APPLICATIONS

This application claims the benefit of the filing date of U.S. Provisional Patent Application No. 63/105,457, filed Oct. 26, 2020, the contents of which are hereby incorporated by reference.

### FEDERALLY SPONSORED RESEARCH AND DEVELOPMENT

This invention was made with Government support of Grant Number 1T32AR073157-01A1 awarded by the National Institutes of Health. The Federal Government has certain rights in this invention.

### BACKGROUND

### 1. Technical Field

The present disclosure relates to methods for treating viral respiratory syndromes, and in particular to methods utilizing an α-2 adrenergic receptor (AR) agonist for treating viral respiratory syndromes.

### 2. Background Information

Coronavirus Disease 2019 (COVID-19) has caused devastating consequences worldwide. No proven treatment exists for severe acute respiratory syndrome coronavirus 2 (SARS-CoV2) mediated Coronavirus Disease 2019 (COVID-19). The predominant cause of death associated with COVID-19 involves respiratory failure from acute respiratory distress syndrome (ARDS)^{1,2}. The progression of ARDS and COVID-19 disease severity is in part mediated through a hyperimmune response. It has been discussed in the literature that an α-2 AR agonist, such as dexmedetomidine, can have immunomodulatory functions, such as reducing inflammation, reducing inflammatory cytokine production, or reducing neutrophil recruitment and activity at the site of an inflammatory stimulus^{3,4}. Dexmedetomidine is an option for sedation in patients with COVID-19 and ARDS requiring mechanical ventilation. No studies to date have assessed dexmedetomidine for the potential to reduce mortality or improve clinical outcomes in patients with COVID-19.

The sedatives most commonly used for intubation and mechanical ventilation include benzodiazepines, propofol, and dexmedetomidine⁵. These sedative medications are often used together at the same time. Furthermore, these sedatives are often combined with an opioid in a sedative-analgesic regimen to optimize patient-ventilator synchrony during invasive mechanical ventilation.

Based on its immunomodulatory function, α-2 AR agonists, such as dexmedetomidine, may be used to inhibit pathways of COVID-19 disease severity and acute respiratory distress syndrome (ARDS) progression and other viral respiratory syndromes.

### BRIEF SUMMARY

Methods for treating viral respiratory syndromes are disclosed. In some aspects, the methods include administering a non-sedative therapeutically effective amount of an α-2 adrenergic receptor (AR) agonist to the subject. In some aspects, the methods include administering a therapeutically effective amount of an α-2 adrenergic receptor (AR) agonist and a therapeutically effective amount of an α-1 adrenergic receptor (AR) antagonist to the subject.

### BRIEF DESCRIPTION OF THE DRAWINGS

FIG. 1 illustrates treatment time points where an α-2 AR agonist may be given to a subject.

FIG. 2 illustrates the mechanism of action of how an α-2 AR agonist works synergistically with an α-1 AR antagonist.

FIG. 3A-3C: Landmark analyses for 28-day mortality at different time cut-off points by multivariable Cox regression accounting for time-varying exposure to dexmedetomidine. 3A) Time to dexmedetomidine (DEX) sedation from time of intubation in the DEX group; 3B) Time to any sedation from time of intubation in the No DEX group. Day 0 includes patients already on respective sedation prior to intubation. 3C) 28-day mortality landmark analysis using Cox regression adjusting for covariates and time varying exposure to DEX from time of intubation. Cut-offs include patients exposed to DEX in the DEX group or any sedation in the No DEX group by days 1-22. All patients included by day 22 time point. FIG. 3A-C illustrates that earlier treatment with an α-2 AR agonist is associated with an even greater reduction in mortality in patients with COVID-19 as opposed to later treatment.

FIG. 4. Treatment with ketamine/xylazine (K/X) anesthesia at the time of intra-articular injection of fMLP at the knee as compared to isoflurane (Iso) anesthesia reduces neutrophil activity at the knee joint as assessed by bioluminescence imaging and quantification of light emission (mean maximum flux, photons/s/cm²/sr) at the region of interest at the knee. Light emission from bioluminescence imaging was assessed prior to surgery and post-operatively (2-6 hours).

FIG. 5. Treatment with ketamine/xylazine (K/X) anesthesia at the time of knee surgery as compared to isoflurane (Iso) anesthesia reduces neutrophil activity at the knee joint as assessed by bioluminescence imaging and quantification of light emission (mean maximum flux, photons/s/cm²/sr) at the region of interest at the knee. Light emission from bioluminescence imaging was assessed prior to surgery and post-operatively (2-6 hours).

### DETAILED DESCRIPTION

The embodiments disclosed below are not intended to be exhaustive or to limit the scope of the disclosure to the precise form in the following description. Rather, the embodiments are chosen and described as examples so that others skilled in the art may utilize its teachings. Unless otherwise defined, all technical and scientific terms used herein have the same meaning as commonly understood by one of ordinary skill in the art to which this disclosure pertains. In case of conflict, the present document, including definitions, will control.

Methods for treating COVID-19 and other viral respiratory syndromes using an α-2 AR agonist are provided. Methods for treating COVID-19 and other viral respiratory syndromes using an α-2 AR agonist and an α-1 AR antagonist are provided.

The uses of the terms “a” and “an” and “the” and similar references (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”, “for example”) provided herein, is intended merely to better illuminate the disclosure and does not pose a limitation on the scope unless otherwise claimed. No language in the specification should be construed as indicating any non-claimed element as essential to the practice of the embodiments.

The term “subject” or “patient” as used herein, refers to a mammal, in some aspects a human.

A “therapeutically effective amount,” “effective dose,” “effective amount,” or “therapeutically effective dosage” of a therapeutic agent, e.g., an alpha-2 adrenergic agonist, is any amount that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The therapeutic agent may inhibit (lessen the severity of or eliminate the occurrence of) and/or prevent a disorder, and/or any one of the symptoms of the disorder. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

“Treating”, “treat”, or “treatment” within the context of the instant disclosure, means an alleviation of symptoms associated with a disorder or disease, or halt of further progression or worsening of those symptoms, or prevention or prophylaxis of the disease or disorder. For example, within the context of this disclosure, successful treatment may include an alleviation of symptoms related to COVID-19 and other viral respiratory syndromes. The treatment may include administering an effective amount of an alpha-2 adrenergic receptor agonist to the subject that results in an alleviation of symptoms associated with a disorder or disease, or halt of further progression or worsening of those symptoms, or prevention or prophylaxis of the disease or disorder.

### Alpha-2 Adrenergic Receptor Agonists

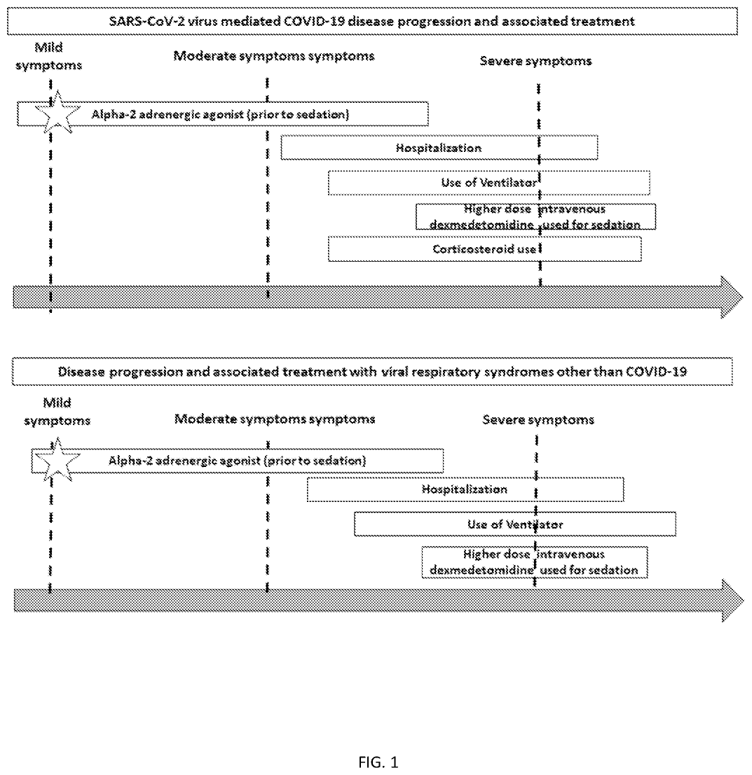

Alpha-2-adrenergic receptor agonists (α2-AR agonists, or α2 agonists) may be delivered to a subject having a viral respiratory syndrome. The α-2 AR agonist may be delivered in a non-sedative amount or a sedative amount. In some embodiments, the α-2 AR agonist may be delivered to a subject who is on a ventilator by intravenous (IV) infusion in an amount that sedates the subject. In other embodiments, the α-2 AR agonist may be delivered in a non-sedative amount to a subject who is hospitalized or not hospitalized and is beginning to exhibit symptoms of a viral respiratory syndrome. The α-2 AR agonist in the non-sedative amount may be administered by any method and in some embodiments may be delivered by other than IV infusion and used on an outpatient basis. Non-limiting examples for suitable administration routes for non-sedative mounts of α-2 AR agonists include intramuscular, intranasal, sublingual/oral and transdermal routes. FIG. 1 illustrates different treatment time points where an α-2 AR agonist may be given in a non-sedative dose beginning with mild symptoms and extending to moderate symptoms for a subject with a viral respiratory syndrome.

α-2 AR agonists such as clonidine, dexmedetomidine, and xylazine are anesthetic agents used widely in clinical and veterinary settings because of their sedative, hypnotic, or analgesic effects. Dexmedetomidine hydrochloride is a highly selective alpha-2 adrenergic agonist with significant sedative, analgesic, and anxiolytic effects. Dexmedetomidine hydrochloride has been approved by the FDA as an IV infusion product (Precedex®) for sedation of initially intubated and mechanically ventilated adult patients in an intensive care setting and is also approved for sedation of non-intubated adult patients as a component of monitored anesthesia care during surgical or diagnostic procedures. Dexmedetomidine is relatively unique in its ability to provide sedation without causing respiratory depression. In some embodiments, the α-2 AR agonist may be dexmedetomidine. In some embodiments, for sedation, dexmedetomidine may be administered by intravenous (IV) infusion at a dose of continuous infusion of 0.2 to 0.7 mcg/kg/hr every 24 hours. Other doses may also be used to sedate the subject.

Dexmedetomidine may also be delivered by intramuscular, intranasal, sublingual/oral and transdermal routes. By way of non-limiting example, dexmedetomidine is marketed as BXL501® for sublingual administration for treatment of agitation and opioid withdrawal symptoms. Alpha-2-adrenergic receptor agonists such as clonidine and tizanidine are available in oral formulations. In some embodiments, dexmedetomidine may be administered in a non-sedative dose. By way of non-limiting example, a non-sedative dose of dexmedetomidine may be delivered by transdermal administration providing about 0.5 mcg/hr to 4 mcg/hour or by IV administration providing about 0.01 mcg/kg/hr to 0.1 mcg/kg/hr.

In some embodiments, the α-2 AR agonist may be clonidine. Clonidine has been used at higher doses for the purpose of sedation, with an effective intravenous infusion dose of approximately 1.2 mg/day. In some embodiments, clonidine may be administered at a non-sedative dose. By way of non-limiting example, clonidine in a non-sedative dose may be administered in a dose range of about 0.1-0.6 mg/day. Other α-2 AR agonists that may be administered in a non-sedative dose include, but are not limited to lofexidine and tizanidine.

In some embodiments, the α-2 AR agonist for use for treatment of viral respiratory syndromes includes, but is not limited to, Dexmedetomidine, Clonidine, Guanfacine, Guanabenz, Guanoxabenz, Guanethidine, Xylazine, Tizanidine, Medetomidine, Methyldopa, Methylnorepinephrine, Fadolmidine, Iodoclonidine, Apraclonidine, Detomidine, Lofexidine, Mivazerol, Azepexol, Talipexol, Rilmenidine, Naphazoline, Tetrahydrozoline, Talipexole, Romifidine, Propylhexedrine, Norfenefrine, Octopamine, Moxonidine, Lidamidine, Tolonidine, UK14304, DJ-7141, ST-91, RWJ-52353, TCG-1000, 4-(3-aminomethyl-cyclohex-3-enylmethyl)-1,3-dihydro-imidazole-2-thione, and 4-(3-hydroxymethyl-cyclohex-3-enylmethyl)-1,3-dihydro-imidazole-2-thione or a pharmaceutically acceptable salt thereof.

In some embodiments, the α-2 AR agonist may be administered in a non-sedative dose. By “non-sedative” is meant that the α-2 AR agonist composition is formulated to deliver an amount of α-2 AR agonist to the subject which does not cause complete sedation of the subject. In other words, a subject remains conscious and responsive throughout the entire time α-2 AR agonist is administered to the subject. In certain instances, throughout administration of the α-2 AR agonist, the subject remains in a cooperative, oriented and tranquil state. In other instances, throughout administration of the α-2 AR agonist, the subject remains alert and capable of responding to commands (e.g., oral or written commands). In yet other instances, throughout administration of the α-2 AR agonist, the subject is in an alert, cooperative, oriented and tranquil state and is capable of responding to commands (e.g., oral or written commands).

Suitable protocols for determining level of sedation may include but are not limited to the Richmond Agitation-Sedation Scale, the Ramsay Sedation Scale, the Vancouver Sedative Recovery Scale, the Glasgow Coma Scale modified by Cook and Palma, the Comfort Scale, the New Sheffield Sedation Scale, the Sedation-Agitation Scale, and the Motor Activity Assessment Scale, among other convenient protocols for determining the level of sedation.

The level of sedation may be evaluated by any convenient protocol, such as with those mentioned above. In certain embodiments, the level of sedation is evaluated using the Richmond Agitation-Sedation Scale (RASS), (as disclosed in Curtis N. Sessler, et al. “The Richmond Agitation-Sedation Scale”, American Journal of Respiratory and Critical Care Medicine, Vol. 166, No. 10 (2002), pp. 1338-1344, the disclosure of which is herein incorporated by reference). For example, each subject may be evaluated by a qualified health care professional and assigned a score for the level of sedation according to the RASS sedation assessment tool.

The Richmond Agitation-Sedation Scale

| Score | Term | Description |
|---|---|---|
| +4 | Combative | Overtly combative or violent; immediate danger to staff |
| +3 | Very agitated | Pulls on or removes tube(s) or catheter(s) or has aggressive |
| | | behavior toward staff |
| +2 | Agitated | Frequent nonpurposeful movement or patient-ventilator |
| | | dyssynchrony |
| +1 | Restless | Anxious or apprehensive but movements not aggressive or |
| | | vigorous |
| 0 | Alert and calm | Spontaneously pays attention to caregiver |
| −1 | Drowsy | Not fully alert, but has sustained (more than 10 seconds) |
| | | awakening, with eye contact, to voice |
| −2 | Light sedation | Briefly (less than 10 seconds) awakens with eye contact to |
| | | voice |
| −3 | Moderate sedation | Any movement (but no eye contact) to voice |
| −4 | Deep sedation | No response to voice, but any movement to physical |
| | | stimulation |
| −5 | Unarousable | No response to voice or physical stimulation |

In some embodiments, during administration of the α-2 AR agonist the level of sedation of a subject is evaluated and the subject is assigned a RASS score. A non-sedative amount of α-2 AR agonist includes a RASS score of 0 or −1. A sedative amount of α-2 AR agonist includes a RASS score of −2 to −5.

### Alpha-1 Adrenergic Receptor Antagonists

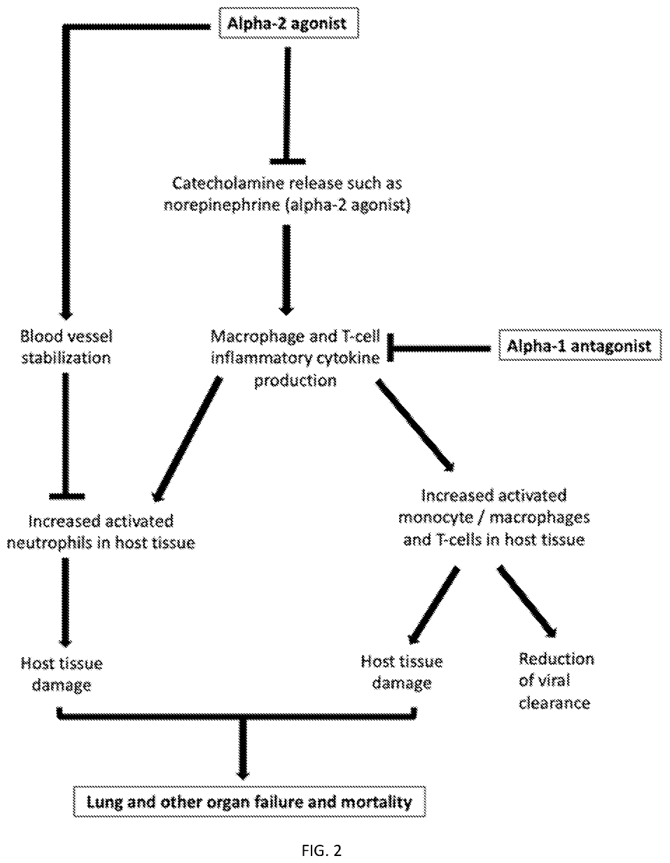

Alpha-1-adrenergic receptor antagonists (α-1 AR antagonists, or α-1 antagonists) may be delivered to a subject having a viral respiratory syndrome. In some embodiments, an α-1 AR antagonist is administered in combination with an α-2 AR agonist. The α-2 AR agonist works synergistically with an α-1 AR antagonist to improve outcomes and reduce mortality in COVID-19 and other viral respiratory syndromes. The mechanism of action is shown in FIG. 2. The α-1 AR antagonist may be administered in combination with a non-sedative dose of the α-2 AR agonist or with a sedative dose of the α-2 AR agonist. The α-1 AR antagonist and the α-2 AR agonist may be administered simultaneously or sequentially in any order.

In some embodiments, the α-1 AR antagonist includes, but is not limited to alfuzosin, dihydroergotamine mesylate, doxazosin, ergotamine, phentolamine mesylate, phenoxybenzamine, prazosin, silodosin, tamsulosin, terazosin, and tolazoline.

### Viral Respiratory Syndromes

The methods disclosed herein relate to treatment of viral respiratory syndromes. In some embodiments, the respiratory virus may be Severe Acute Respiratory Syndrome Coronavirus 2 (SARS-CoV-2). In other embodiments, the respiratory virus may include, but is not limited to, Rhinoviruses, influenza (Flu A and B), adenoviruses, picornaviruses, respiratory syncytial virus, parainfluenza viruses 1-3, human metapneumovirus, coronaviruses (other than SARS-CoV-2, including Middle East Respiratory Syndrome Coronavirus (MERS-CoV), Severe Acute Respiratory Syndrome Coronavirus (SARS-CoV)) and the herpesviruses.

### Pharmaceutical Compositions

The α-2 AR agonists and/or the α-1 AR antagonists described herein may be used alone or in compositions together with a pharmaceutically acceptable carrier or excipient. Pharmaceutical compositions of the disclosed herein may comprise a therapeutically effective amount of one or more α-2 AR agonists and/or the α-1 AR antagonists, together with one or more pharmaceutically acceptable carriers. In some embodiments, the each α-2 AR agonist and/or each α-1 AR antagonist is used alone with a pharmaceutically acceptable carrier or excipient such that no other active agent is administered.

As used herein, the term “pharmaceutically acceptable carrier” means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringers solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. Other suitable pharmaceutically acceptable excipients are described in “Remington's Pharmaceutical Sciences,” Mack Pub. Co., New Jersey, 1991, incorporated herein by reference.

The α-2 AR agonists and/or the α-1 AR antagonists described herein may be administered to humans and animals in dosage unit formulations containing conventional nontoxic pharmaceutically acceptable carriers, adjuvants, and vehicles as desired.

Methods of formulation are well known in the art and are disclosed, for example, in Remington: The Science and Practice of Pharmacy, Mack Publishing Company, Easton, Pa., 19th Edition (1995). Pharmaceutical compositions for use in the present disclosure can be in the form of sterile, non-pyrogenic liquid solutions or suspensions, coated capsules or lipid particles, lyophilized powders, or other forms known in the art.

Liquid dosage forms for oral administration include pharmaceutically acceptable emulsions, microemulsions, solutions, suspensions, syrups and elixirs. In addition to the α-2 AR agonists and/or the α-1 AR antagonists, the liquid dosage forms may contain inert diluents commonly used in the art such as, for example, water or other solvents, solubilizing agents and emulsifiers such as ethyl alcohol, isopropyl alcohol, ethyl carbonate, EtOAc, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Solid dosage forms for oral administration include capsules, tablets, pills, powders, and granules. In such solid dosage forms, the α-2 AR agonists and/or the α-1 AR antagonists is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, acetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

The α-2 AR agonists and/or the α-1 AR antagonists can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the α-2 AR agonists and/or the α-1 AR antagonists may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of the α-2 AR agonists and/or the α-1 AR antagonists include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulations, ear drops, and the like are also contemplated as being within the scope of this disclosure.

The ointments, pastes, creams and gels may contain, in addition to the α-2 AR agonist and/or the α-1 AR antagonist, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

The α-2 AR agonists and/or the α-1 AR antagonists may also be formulated for use as topical powders and sprays that can contain, in addition to the α-2 AR agonists and/or the α-1 AR antagonists, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches may also be used for providing controlled delivery of the α-2 AR agonists and/or the α-1 AR antagonists to the body. Such dosage forms can be made by dissolving or dispensing the α-2 AR agonists and/or the α-1 AR antagonists in the proper medium. Absorption enhancers can also be used to increase the flux of the α-2 AR agonist and/or the α-1 AR antagonist across the skin. The rate can be controlled by either providing a rate controlling membrane or by dispersing the α-2 AR agonist and/or the α-1 AR antagonist in a polymer matrix or gel. The α-2 AR agonists and/or the α-1 AR antagonists can also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes can be used. The present compositions in liposome form can contain, in addition to the α-2 AR agonist and/or the α-1 AR antagonist, stabilizers, preservatives, excipients, and the like. The preferred lipids are the phospholipids and phosphatidyl cholines (lecithins), both natural and synthetic. Methods to form liposomes are known in the art. See, for example, Prescott (ed.), “Methods in Cell Biology,” Volume XIV, Academic Press, New York, 1976, p. 33 et seq.

The α-2 AR agonists and/or the α-1 AR antagonists as used herein may be formulated for delivery as a liquid, aerosol or inhalable dry powder. α-2 AR agonists and/or the α-1 AR antagonists may be delivered nasally or by inhalation. Liquid aerosol formulations may be nebulized predominantly into particle sizes that can be delivered to the terminal and respiratory bronchioles.

Aerosolized formulations of the α-2 AR agonists and/or the α-1 AR antagonists described herein may be delivered using an aerosol forming device, such as a jet, vibrating porous plate or ultrasonic nebulizer, preferably selected to allow the formation of an aerosol particles having with a mass medium average diameter predominantly between 1 to 5 μm. Further, the formulation preferably has balanced osmolarity ionic strength and chloride concentration, and the smallest aerosolizable volume able to deliver effective dose of the α-2 AR agonist and/or the α-1 AR antagonist to the site of treatment. Additionally, the aerosolized formulation preferably does not impair negatively the functionality of the airways and does not cause undesirable side effects.

Aerosolization devices suitable for administration of aerosol formulations of the α-2 AR agonists and/or the α-1 AR antagonists include, for example, jet, vibrating porous plate, ultrasonic nebulizers and energized dry powder inhalers, that are able to nebulize the formulation of the α-2 AR agonists and/or the α-1 AR antagonists into aerosol particle size predominantly in the size range from 1-5 μm. Predominantly in this application means that at least 70% but preferably more than 90% of all generated aerosol particles are within 1-5 μm range. A jet nebulizer works by air pressure to break a liquid solution into aerosol droplets. Vibrating porous plate nebulizers work by using a sonic vacuum produced by a rapidly vibrating porous plate to extrude a solvent droplet through a porous plate. An ultrasonic nebulizer works by a piezoelectric crystal that shears a liquid into small aerosol droplets. A variety of suitable devices are available, including, for example, AERONEB and AERODOSE vibrating porous plate nebulizers (AeroGen, Inc., Sunnyvale, California), SIDESTREAM nebulizers (Medic-Aid Ltd., West Sussex, England), PARI LC and PARI LC STAR jet nebulizers (Pari Respiratory Equipment, Inc., Richmond, Virginia), and AEROSONIC (DeVilbiss Medizinische Produkte (Deutschland) GmbH, Heiden, Germany) and ULTRAAIRE (Omron Healthcare, Inc., Vernon Hills, Illinois) ultrasonic nebulizers.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-propanediol or 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form may be accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations may also be prepared by entrapping the drug in liposomes or microemulsions, which are compatible with body tissues.

The α-2 AR agonists and/or the α-1 AR antagonists described herein can be administered alone or in combination with other agents, for a possible combination therapy being staggered or given independently of one another. Long-term therapy is equally possible as is adjuvant therapy in the context of other treatment strategies, as described above. Other possible treatments are therapy to maintain the patient's status after the initial treatment, or even preventive therapy, for example in patients at risk.

Effective amounts of the α-2 AR agonists and/or the α-1 AR antagonists generally include any amount sufficient to detectably an inhibition or alleviation of symptoms. The amount of active ingredient that may be combined with the carrier materials to produce a single dosage form will vary depending upon the host treated and the particular mode of administration. It will be understood, however, that the specific dose level for any particular patient will depend upon a variety of factors including the activity of the specific α-2 AR agonist and/or the α-1 AR antagonist employed, the age, body weight, general health, sex, diet, time of administration, route of administration, rate of excretion, drug combination, and the severity of the particular disease undergoing therapy. The therapeutically effective amount for a given situation can be readily determined by routine experimentation and is within the skill and judgment of the ordinary clinician.

If the α-2 AR agonist and/or the α-1 AR antagonist is administered in combination with another compound, the term “amount that is effective” is understood to mean that amount of α-2 AR agonist and/or the α-1 AR antagonist in combination with the additional compound to achieve the desired effect. In other words, a suitable combination therapy according to the current disclosure encompasses an amount of the α-2 AR agonist and/or the α-1 AR antagonist and an amount of the additional compound, either of which when given alone at that particular dose would not constitute an effective amount, but administered in combination would be an “amount that is effective”.

It will be understood, however, that the total daily usage of the α-2 AR agonist and/or the α-1 AR antagonist and compositions of the present disclosure will be decided by the attending physician within the scope of sound medical judgment. The specific therapeutically effective dose level for any particular patient will depend upon a variety of factors including the disorder being treated and the severity of the disorder; the activity of the specific α-2 AR agonist and/or the α-1 AR antagonist employed; the specific composition employed; the age, body weight, general health, sex and diet of the patient; the time of administration, route of administration, and rate of excretion of the specific α-2 AR agonist and/or the α-1 AR antagonist employed; the duration of the treatment; drugs used in combination or coincidental with the specific α-2 AR agonist and/or the α-1 AR antagonist employed; and like factors well known in the medical arts.

### EXAMPLE 1

### Baseline Characteristics

From Mar. 1, 2020 to Jul. 30, 2020, a total of 214 patients met criteria for the analysis. A total of 152 patients were in the dexmedetomidine (DEX) group. The remaining patients (n=62) were in the No DEX group. Patient demographic characteristics and comorbidities at hospital admission were evaluated between groups (Table 1). Age was similar between the DEX and No DEX group (60.1 vs. 59.1 years; p=0.83). Gender, race, and ethnicity were similar between groups. Within the DEX group, there was a higher proportion of patients with hypertension (78.9% vs. 61.3%; p=0.008) and coronary artery disease (23.0% vs. 11.3%; p=0.05). Active cancer and types of chronic respiratory disease, immunosuppression, kidney disease, liver disease, and metabolic disease were similar between groups. BMI was similar in the DEX group and No DEX group (33.5 vs. 34.7 kg/m²; p=0.36). The mCCI trended higher but was not statistically significant in the DEX group vs. No DEX group (2.5 vs. 1.8; p=0.07) (Table 1). Pao₂/Flo₂ values were similar between the DEX Group and No DEX Group at time of intubation (132.7 vs. 122.8 mmHg; p=0.40). The mSOFA scores were similar between the DEX Group and No DEX Group at time of intubation (8.0 vs. 8.3; p=0.55).

### Primary and Secondary Outcomes

From start time of intubation, 28-day mortality in the cohort receiving DEX was 27.0% as compared to 64.5% in the cohort that did not receive DEX (relative risk reduction 58.2%; 95% CI 42.4-69.6%). The use of DEX was associated with reduced 28-day mortality on multivariable Cox regression analysis from time of intubation (aHR, 0.19; 95% CI, 0.10 to 0.33; p<0.001). The use of DEX was also associated with reduced 28-day mortality on unadjusted univariate Cox regression analysis from time of intubation (HR, 0.25; 95% CI, 0.16 to 0.39; p<0.001).

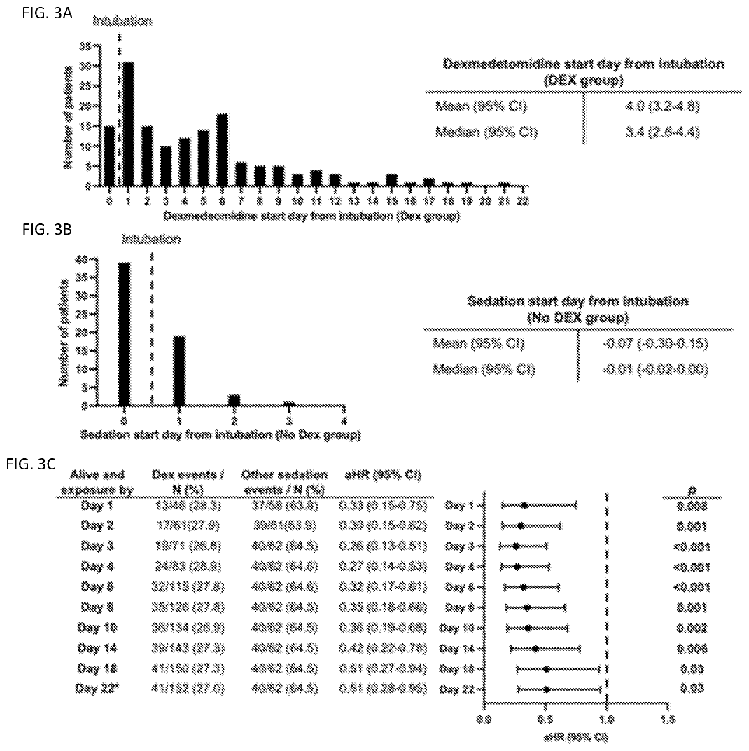

DEX was often started days post intubation (mean 4.0 days, median 3.4 days) (FIG. 3A). Multivariable Cox regression assessing for 28-day mortality from time of intubation adjusting for time varying exposure to DEX revealed that DEX use was associated with reduced mortality (aHR, 0.51; 95% CI 0.28 to 0.95; p=0.03); landmark analysis with different time cut-offs (days 1-22) for start of DEX use (DEX group) or any sedation use (No DEX group) demonstrated that earlier DEX use overall from time of intubation was associated with lower aHRs and increased significance (FIG. 3C). As comparison, univariable Cox regression assessing for 28-day mortality from time of intubation adjusting for time varying exposure to DEX also demonstrated that DEX use was associated with reduced mortality (aHR, 0.56; 95% CI 0.35 to 0.91; p=0.02). In all adjusted and unadjusted analyses performed, DEX use was associated with reduce 28-day mortality from time of intubation (Table 2).

### Additional Analyses

As an additional and supplementary measure, we discovered in patients with the highest time on DEX relative to period of time they were on invasive mechanical ventilation (90% or greater), patients had lower in-hospital mortality, lower time on intubation and mechanical ventilation, improved or higher ventilator free days at day 60 and improved or higher ICU fee days at day 60 (Table 3).

### Discussion

We performed a retrospective analysis to assess mortality associated with α₂ AR agonist use in patients with COVID-19. To achieve this objective, we evaluated use of DEX (an ICU sedative) in critically ill patients with COVID-19 requiring sedation and invasive mechanical ventilation. DEX use was associated with reduced 28-day mortality from time of intubation on all multivariable and univariable Cox regression analyses. Landmark analysis demonstrated that earlier DEX use was associated with a further reduction in mortality

On additional analyses, we found a greater proportion of use of dexmedetomidine relative to time intubation and on mechanical ventilation was associated with reduced in-hospital mortality, reduced time on intubation and mechanical ventilation, higher ventilator free days at day 60, and higher ICU free days at day 60.

Recent evidence suggests that corticosteroids reduce mortality in critically ill patients with COVID-19^{7,8}. Corticosteroids are a powerful class of immunosuppressants that have broad functions of suppressing inflammatory cytokine production as well as immune cell activity, including neutrophils, B and T lymphocytes, monocytes, and macrophages. A potential drawback in corticosteroids in the setting of COVID-19, infection, and injury to the lungs is suppression of host immune response to viral infection as well as recovery from tissue damage. A review of human and animal studies has shown overall that α2 receptor agonist, such dexmedetomidine or clonidine, has an immunomodulatory function that may be directly (i.e., monocyte/macrophage function; central sympatholytic effects, stimulation of cholinergic pathways) or indirectly (i.e., indirect sympatholytic response) associated with α2 receptor agonism, which can overall be associated with reduced inflammatory cytokine production⁴. Results generated from in vitro and animal studies have also revealed α2 receptor agonists can prevent recruitment of neutrophils to the site of inflammatory stimulus; this may be through direct action on leukocytes or stabilization of blood vessel vascular endothelial cells in an inflammatory environment³; we speculate this may be the reason for improved outcomes in critically ill patients with COVID-19. Furthermore, by directly targeting neutrophil activity, which has a defined role in ARDS progression⁹ and has been associated with COVID-19 disease severity¹⁰⁻¹², and having less effect on the entire immune response as compared to corticosteroids, we speculate dexmedetomidine likely causes less suppression of the host immune response to viral infection.

Our patient population was a single hospital system. Larger studies in the future may allow for better assessment. Our study is limited in being retrospective. There was no standardization of sedative received. Patients receiving dexmedetomidine had large variances in terms of how long they received the drug and when they received the drug. Individual patient dosing of dexmedetomidine was not standardized between patients and drug administration was individualized based on clinician order. A randomized controlled trial with early and persistent use of dexmedetomidine compared to no dexmedetomidine is warranted given the findings of this study.

In conclusion, use of dexmedetomidine was associated with lower mortality and improved clinical outcomes. Dexmedetomidine could be an important sedative or pharmacologic agent in patients with COVID-19 to reduce mortality and improve clinical outcomes.

### Methods

### Data Sources

Data was collected from electronic medical records (EMRs) of Rush University hospitals: Rush University Medical Center; Rush Copley Medical Center; and Rush Oak Park Hospital. Deidentified data was collected from the EMRs by the Rush Bioinformatics and Biostatistics Core. This retrospective cohort study received expedited approval by the Institutional Review Board at Rush University Medical Center. All authors analyzed the data.

### Study Population

We identified patients that were hospitalized at Rush University from Mar. 1, 2020 to Jul. 30, 2020. Hospitalized patients were included if they were of adult age (≥18), had a diagnosis of COVID-19, had ARDS or related diagnosis, and received intubation. Of this population, we excluded patients that had a diagnosis of autoimmune disease or if tocilizumab was ordered/administered during hospital admission; these patients were excluded because there were a large number of patients at Rush receiving tocilizumab, and both autoimmune disease or associated prescribed medications or tocilizumab can alter the immune system and immune response. Corticosteroids were not excluded because at the time of the study there were no standard practices or guidelines for using this medication in the study population at Rush. Clinical outcomes were assessed from the time of admission to the time of discharge. We formulated sedation groups as follows: i) dexmedetomidine group (DEX Group): use of dexmedetomidine with or without any other sedative; ii) no dexmedetomidine group (No DEX Group): use of any sedative measure other than dexmedetomidine.

We evaluated patients in each group for age, gender, race, ethnicity, and comorbidities at admission. We also calculated a modified Charlson Comorbidity Index (CCI) at admission score as described by Quan et al., 2011⁶. Directly prior to intubation, a sequential organ failure assessment score (SOFA) was calculated for each group, and a PaO₂/FiO₂ (mmHg) was separately assessed.

### Study End Points

We assessed 28-day mortality between the DEX and No DEX groups from start time of intubation. Our primary tool to assess 28-day mortality was multivariable Cox proportional hazards regression. Within this Cox regression model, DEX and covariates chosen a priori based on greatest potential influence on mortality were included. Covariates besides DEX included the following: i) age at hospital admission; ii) body mass index (BMI) at hospital admission; iii) mCCI at hospital admission; iv) Pao₂/Flo₂ at intubation; v) mSOFA at intubation; vi) corticosteroid use; vii) prone positioning use.

In addition, 28-day mortality between the DEX and No DEX groups using Cox proportional hazards regression accounting for time varying exposure to the drug under investigation (DEX) from time of intubation adjusting for immortal time bias was performed; covariates addressed above, chosen a priori for potential influence on mortality, were also included. To get a better understanding if timing of DEX initiation influenced mortality, we performed a landmark analysis, assessing different time cut-off points based on start time of the sedative DEX use (DEX group) or start time of any sedation use other than DEX (No DEX group) from time of intubation, using Cox proportional hazard regression accounting for time varying exposure to DEX.

As an additional analysis, we investigate duration of dexmedetomidine use relative to time on intubation in relation to in-hospital mortality, time on intubation and mechanical ventilation, ICU time, hospital length of stay, ventilator free days at day 60, ICU free days at day 60.

### Statistical Analysis

For continuous variables, independent samples t-tests were performed. For each continuous variable, a Levene's test for equality of variances was performed. With significance on Levene's test for heterogeneity of variances, a Mann-Whitney U test was performed. Continuous variable data are displayed as mean±95% confidence interval (CI). Categorical variables were assessed with a Pearson's chi-squared test. If any expected count in a 2×2 table was less than 5, a Fisher's exact test was performed. Categorical variables are displayed as counts and calculated as percentage within the group. We used p≤0.05 as the threshold for significance.

Mortality outcomes for DEX use and other covariates were evaluated with adjusted hazard ratios (aHR) with respective 95% CI and p≤0.05 for significance. Simple imputation using the mean of the immediate preceding and succeeding most severe value over 24 hours was used for missing values for Pao₂/Flo₂ and mSOFA scores within the 24 hour time period of interest (intubation) (11-13). A complete case analysis was performed (14). All analyses were performed using SPSS version 27.

TABLE 1
Patient Baseline Characteristics at Hospital Admission

| | Dexmedetomidine | No Dexmedetomidine | |
|---|---|---|---|
| Characteristics | n = 152 | n = 62 | p |
| Age | 60.1 (58.1-62.2) | 59.1 (54.9-63.2) | 0.83 |
| Male sex | 95 (62.5%) | 38 (61.3%) | 0.87 |
| Race | | | |
| American | 0 (0.00%) | 0 (0.00%) | >0.999 |
| Asian | 5 (3.3%) | 1 (1.6%) | 0.67 |
| Black or | 52 (34.2%) | 27 (43.5%) | 0.20 |
| White | 38 (25.0%) | 15 (24.2%) | 0.90 |
| Other / Not | 57 (37.5%) | 19 (30.6%) | 0.34 |
| Ethnicity | | | |
| Hispanic or | 66 (43.4%) | 20 (32.3%) | 0.13 |
| Not Hispanic | 84 (55.3%) | 40 (64.5%) | 0.21 |
| Other / Not | 2 (1.3%) | 2 (3.2%) | 0.58 |
| Active Cancer | 13 (8.6%) | 5 (8.1%) | 0.91 |
| Cardiovascular disease | | | |
| Hypertension | 120 (78.9%) | 38 (61.3%) | 0.008 |
| Coronary | 35 (23.0%) | 7 (11.3%) | 0.05 |
| Congestive | 38 (25.0%) | 14 (22.6%) | 0.71 |
| Chronic respiratory disease | | | |
| Asthma | 13 (8.6%) | 7 (11.3%) | 0.53 |
| COPD | 25 (16.4%) | 10 (16.1%) | 0.95 |
| Interstitial | 7 (4.6%) | 1 (1.6%) | 0.44 |
| Obstructive | 21 (13.8%) | 10 (16.1%) | 0.66 |
| Immunosuppression | | | |
| HIV | 1 (0.66%) | 0 (0.00%) | >0.999 |
| History of | 3 (2.0%) | 3 (4.8%) | 0.36 |
| Kidney disease | | | |
| Chronic | 49 (32.2%) | 13 (21.0%) | 0.10 |
| End-stage | 15 (9.9%) | 4 (6.5%) | 0.43 |
| Liver disease | | | |
| Cirrhosis | 7 (4.6%) | 1 (1.6%) | 0.44 |
| Chronic | | | |
| Hepatitis B | 0 (0.00%) | 0 (0.00%) | >0.999 |
| Hepatitis C | 1 (0.66%) | 0 (0.00%) | >0.999 |
| Metabolic disease | | | |
| Obesity (BMI | 65 (42.8%) | 27 (43.5%) | 0.92 |
| Morbid | 27 (17.8%) | 15 (24.2%) | 0.28 |
| BMI | 33.5 (32.1-34.9) | 34.7 (32.4-37.0) | 0.36 |
| Diabetes | 70 (46.1%) | 25 (40.3%) | 0.44 |
| Modified | 2.5 (2.1-2.9) | 1.8 (1.2-2.4) | 0.07 |

Continuous variables represented by mean (95% CI) with p-values represented by independent samples t-test or Mann-Whitney U test as appropriate;
categorical variables represented by count and (%) of group with p-values represented by Pearson's chi-squared test or Fisher’s exact test as appropriate.
COPD = chronic obstructive pulmonary disease;
HIV = human immunodeficiency virus;
BMI = body mass index

TABLE 2
28-Day Mortality from Time of Intubation

| Characteristics | HR | aHR | p |
|---|---|---|---|
| Multivariable† Cox regression (DEX use) | — | 0.19 (0.10- [symbol] | <0.001 |
| Univariable Cox Regression (DEX use) | 0.25 (0.16-0.39) | — | <0.001 |
| Multivariable† Cox Regression (DEX use) | — | 0.51 (0.28- | 0.03 |
| with DEX use at a time-varying covariate | | 0.95) | |
| Univariable Cox Regression (DEX use) | — | 0.56 (0.35 | 0.02 |
| with DEX use at a time-varying covariate | | to 0.91) | |

Values represented by hazard ratio (HR) or adjusted hazard ratio (aHR) (95% CI)
†Variables in multivariable analysis include: i) DEX use ii) age at hospital admission; iii) body mass index (BMI) at hospital admission; iv) mCCI at hospital admission; v) Pao2/F102 at intubation; vi) mSOFA at intubation; vi) corticosteroid use; vii) prone positioning use
DEX: dexmedetomidine [symbol]
indicates data missing or illegible when filed

TABLE 3
Clinical Outcomes Based on Proportion of Time on Dexmedetomidine
Relative to Time on Intubation (Time on Dexmedetomidine/Time on Intubation)

| | | | Risk Ratio^{#} or |
|---|---|---|---|
| | | | Difference |
| | 90% or greater | Less than 90% | Between Groups |
| | (n = 30) | (n = 122) | (95% CI) |
| In-hospital mortality (%) | 3/30 | 44/122 | 0.277 |
| | (10.0%) | (36.1%) | (0.0924-0.832)* |
| Intubation time (days) | 9.61 | 15.53 | −5.92 |
| | (7.00-12.23) | (14.15-16.91) | (−8.97 to −2.87) |
| ICU time (days) | 18.76 | 20.59 | −1.83 |
| | (14.32-23.20) | (18.39-22.79) | (−6.74-3.08) |
| Hospital length of stay (days) | 24.37 | 25.75 | −1.38 |
| | (19.99-28.75) | (23.44-28.05) | (−6.47-3.71) |
| Ventilator free days at day 60 | 43.63 | 28.08 | 15.55 |
| | (37.32-49.95) | (24.09-32.08) | (8.17-22.94) |
| ICU Free days at day 60 | 36.45 | 24.98 | 11.48 |
| | (30.13-42.78) | (21.19-28.77) | (4.19-18.77) |

*Mean values represented with ± 95% CI; counts represented as percent
# Value represented as risk ratio with 95% CI

### EXAMPLE 2

Prevention of COVID-19 disease progression and mortality with early treatment with non-sedative dose alpha-2 adrenergic receptor (α2-AR) agonist

Study type: Interventional study, prospective randomized clinical trial

Intervention/treatment:

Experimental group: α2-AR agonist

Patients will receive an α2-AR agonist daily for 30 days

Oral clonidine 0.2 mg/day [0.1 mg PO twice a day] to 0.6 mg/day [0.3 mg PO twice a day]

Control group: standard of care

Inclusion criteria i) Tested positive for SARS-CoV-2; ii) Clinical symptoms of COVID-19

Exclusion criteria i) At initial enrollment, requirement of oxygenation beyond high-flow nasal canula oxygenation (e.g., noninvasive positive pressure ventilation or intubation/invasive mechanical ventilation). ii) Level of care necessary for intensive care unit (ICU)/critical care unit (CCU) admittance. Iii) Standard contraindications/precautions to α2-AR agonist clonidine (e.g., atrioventricular (AV) block, bradycardia, cerebrovascular disease, dehydration, heart failure, hypotension, myocardial infarction, orthostatic hypotension, syncope).

Primary outcomes: outcomes will be assessed over 60 days from period of enrollment i) Death, ii) Requirement of hospitalization on invasive mechanical ventilation or ECMO (extracorporeal membrane oxygenation) or level of care necessary for admittance to ICU/CCU. iii) Requirement of hospitalization on non-invasive ventilation or high flow nasal canula. iv) Requirement of hospitalization on supplemental oxygen. v) Requirement of hospitalization not on supplemental oxygen. vi) Cumulative incidence of serious adverse events. vii) Cumulative incidence of symptomatic hypotension.

### EXAMPLE 3

Prevention of COVID-19 disease progression and mortality with early treatment with non-sedative dose alpha-2 adrenergic receptor (α2-AR) agonist

Study type: Interventional study, prospective randomized clinical trial

Intervention/treatment:

Experimental group: α2-AR agonist

Patients will receive an α2-AR agonist daily for 30 days

Transdermal dexmedetomidine 0.5 mcg/hr to 4 mcg/hr

Control group: standard of care

Inclusion criteria i) Tested positive for SARS-CoV-2; ii) Clinical symptoms of COVID-19

Exclusion criteria i) At initial enrollment, requirement of oxygenation beyond high-flow nasal canula oxygenation (e.g., noninvasive positive pressure ventilation or intubation/invasive mechanical ventilation). ii) Level of care necessary for intensive care unit (ICU)/critical care unit (CCU) admittance. Iii) Standard contraindications/precautions to α2-AR agonist clonidine (e.g., atrioventricular (AV) block, bradycardia, cerebrovascular disease, dehydration, heart failure, hypotension, myocardial infarction, orthostatic hypotension, syncope).

Primary outcomes: outcomes will be assessed over 60 days from period of enrollment i) Death, ii) Requirement of hospitalization on invasive mechanical ventilation or ECMO (extracorporeal membrane oxygenation) or level of care necessary for admittance to ICU/CCU. iii) Requirement of hospitalization on non-invasive ventilation or high flow nasal canula. iv) Requirement of hospitalization on supplemental oxygen. v) Requirement of hospitalization not on supplemental oxygen. vi) Cumulative incidence of serious adverse events. vii) Cumulative incidence of symptomatic hypotension.

### EXAMPLE 4

Prevention of COVID-19 disease progression and mortality with early treatment with a combined non-sedative dose alpha-2 adrenergic receptor (α2-AR) agonist and alpha-1 adrenergic receptor (α1-AR) antagonist

Study type: Interventional study, prospective randomized clinical trial

Intervention/treatment:

Experimental group: α2-AR agonist+α1-AR antagonist

Patients will receive a combined dose of an α2-AR agonist and α1-AR antagonist daily for 30 days

α2-AR agonist and dose: oral clonidine 0.2 mg/day [0.1 mg PO twice a day] to 0.6 mg/day [0.3 mg PO twice a day]

α1-AR antagonist and dose: oral prazosin 1.5 mg/day-15 mg/day [0.5 mg to 5 mg per 8 hours]

Control group: standard of care

Inclusion criteria i) Tested positive for SARS-CoV-2; ii) Clinical symptoms of COVID-19

Exclusion criteria i) At initial enrollment, requirement of oxygenation beyond high-flow nasal canula oxygenation (e.g., noninvasive positive pressure ventilation or intubation/invasive mechanical ventilation). ii) Level of care necessary for intensive care unit (ICU)/critical care unit (CCU) admittance. Iii) Standard contraindications/precautions to α2-AR agonist clonidine or α1-AR antagonist prazosin (e.g., atrioventricular (AV) block, bradycardia, cerebrovascular disease, dehydration, heart failure, hypotension, myocardial infarction, orthostatic hypotension, syncope).

Primary outcomes: outcomes will be assessed over 60 days from period of enrollment i) Death, ii) Requirement of hospitalization on invasive mechanical ventilation or ECMO (extracorporeal membrane oxygenation) or level of care necessary for admittance to ICU/CCU. iii) Requirement of hospitalization on non-invasive ventilation or high flow nasal canula. iv) Requirement of hospitalization on supplemental oxygen. v) Requirement of hospitalization not on supplemental oxygen. vi) Cumulative incidence of serious adverse events. vii) Cumulative incidence of symptomatic hypotension.

### EXAMPLE 5

Treatment of moderate to severe COVID-19 symptoms with combined treatment sedative dose alpha-2 adrenergic receptor (α2-AR) agonist and alpha-1 adrenergic receptor (α1-AR) antagonist

Study type: Interventional study, prospective randomized clinical trial

Intervention/treatment:

Experimental group: α2-AR agonist+α1-AR antagonist

Patients will receive a combined dose of an α2-AR agonist and α1-AR antagonist during sedation for patients on invasive mechanical ventilation

α2-AR agonist: IV continuous infusion of dexmedetomidine 0.2 to 0.7 mcg/kg/hour IV every 24 hours

α1-AR antagonist: IV continuous infusion of prazosin 0.5 to 10 mcg/kg/hour IV every 24 hours

Inclusion criteria i) Tested positive for SARS-CoV-2; ii) Clinical symptoms of COVID-19 iii) Requirement of intubation/invasive mechanical ventilation

Exclusion criteria i) Contraindications to α2-AR agonist dexmedetomidine or α1-AR antagonist prazosin (e.g., atrioventricular (AV) block, bradycardia, hypotension)

Primary outcomes: outcomes will be assessed over 60 days from period of enrollment i) Death. ii) ICU-Free days (28-days and 60-days). iii) Ventilator-Free days (28-days and 60-days). iv) Cumulative incidence of serious adverse events. v) Cumulative incidence of symptomatic hypotension.

### EXAMPLE 6

Treatment with a mixture of ketamine and alpha-2 adrenergic receptor agonist xylazine suppresses neutrophil recruitment and activity at the knee joint in response to a pathogen association molecular pattern (PAMP) immune cell activator N-formyl-methionyl-leucyl-phenylalanine (fMLP) and in response to damage associated molecular patterns (DAMPs) from knee joint surgery.

### Results

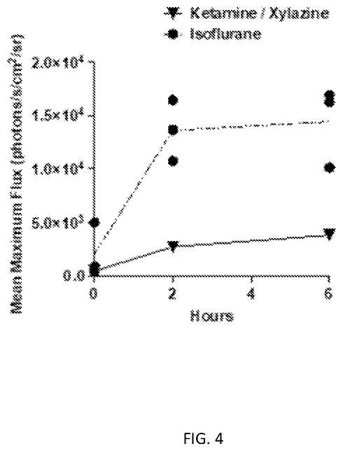

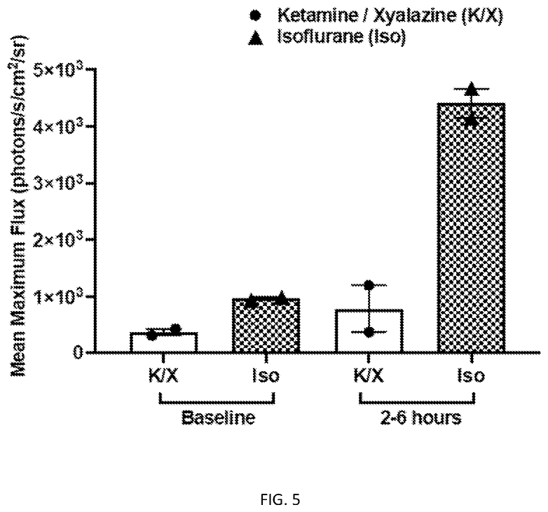

We compared the neutrophil response to pathogen associated molecular pattern (PAMP) and damaged associated molecular patterns (DAMPs) in response to sedation with injection mixture of ketamine (90 mg/kg) and alpha-2 adrenergic receptor agonist xylazine (4.5 mg/kg) or 2% isoflurane gas anesthesia and compared neutrophil activity between the two sedation group with in vivo bioluminescence imaging. Use of injection mixture of ketamine (90 mg/kg) and xylazine (4.5 mg/kg) resulted: i) suppressed neutrophil activity at the knee joint in response local application of the PAMP (N-formyl-methionyl-leucyl-phenylalanine, abbreviated as fMLP) (FIG. 4); ii) suppressed neutrophil activity at the knee joint in response to local creation of DAMPs by tissue trauma created by surgery (FIG. 5).

### Discussion

To evaluate the immune response, we evaluated neutrophil activity in response: i) local application at the knee joint with a powerful mediator of the innate immune response and neutrophil activity (N-formyl-methionyl-leucyl-phenylalanine, abbreviated as fMLP) and ii) Tissue trauma/damage at the knee joint by performing a surgical procedure (surgical placement of a bone intramedullary implant). The peptide (fMLP) is a pathogen associated molecular pattern (PAMP) that activates neutrophils and other immune cells. Tissue trauma caused by surgery releases damage associated molecular patterns (DAMPS); similar to PAMPS, DAMPS stimulate activation of neutrophils and other immune cells.

Our animal studies demonstrate that use of ketamine (90 mg/kg) and xylazine (4.5 mg/kg) as opposed 2% isoflurane gas led to substantially diminished neutrophil activity at the knee joint. This effect was seen in response to PAMP fMLP as well as DAMPs created by surgery.

Viral RNA serves as a PAMP that stimulates the immune and inflammatory response. SARS-related coronaviruses have a number of components that serve as PAMPS include viral RNA and S protein¹³. Furthermore, viral infection, for instance with SARS-CoV-2, causes cell necrosis and tissue damage leading to release of DAMPs perpetuating a hyperimmune and hyperinflammatory state¹⁴.

Since, in particular, COVID-19 progression and mortality is associated with a hyperinflammatory state and substantial response to PAMPS and DAMPS, further research should investigate whether an alpha-2 agonist, such as xylazine, or ketamine can reduce COVID-19 disease progression and mortality.

### Methods

### Study Overview

To evaluate the immune response, we evaluated neutrophil activity in response: i) local application at the knee joint with a powerful mediator of the innate immune response and neutrophil activity (N-formyl-methionyl-leucyl-phenylalanine, abbreviated as fMLP) and ii) Tissue trauma/damage at the knee joint by performing a surgical procedure (surgical placement of a bone intramedullary implant). The peptide (fMLP) is a pathogen associated molecular pattern (PAMP) that activates neutrophils and other immune cells. Tissue trauma caused by surgery releases damage associated molecular patterns (DAMPS); similar to PAMPS, DAMPS stimulates activation of neutrophils and other immune cells.

### Animal Experimentation

We have an approval from the Rush University Medical Center Institutional Animal Care and Use Committee (IACUC No: 19-623) to conduct research as indicated. All procedures complied strictly with the standards for care and use of animal subjects as stated in the Guide for the Care and Use of Laboratory Animals (Institute of Laboratory Animal Resources, National Academy of Sciences, Bethesda, MD, USA). We obtained C57BL/6J mice from the Jackson Laboratories (Bar Harbor, ME). These mice were allowed to acclimate to the environment for 1 week prior to experimentation.

### Intra-Articular Injection

To demonstrate that fMLP is capable of mobilizing and directing neutrophil response in the knee joint even in the absence of infection and/or injury, 12-week-old C57BL/6J mice (Jackson Laboratory) received local intra-articular injection of fMLP (Sigma Aldrich, F3506) or vehicle control at the right knee joint. Prior to injection, right hindlimbs were shaved and scrubbed with 70% ethanol followed by iodine. fMLP was prepared at a concentration of (1 μg/μL) in a solution of 25% dimethylsulfoxide (DMSO) and 75% phosphate buffered saline (PBS). Vehicle control was a 5 μL solution of 25% DMSO and 75% PBS. Mice were anesthetized with 2% isoflurane gas anesthesia or an injection mixture of ketamine (90 mg/kg) and xylazine (4.5 mg/kg). A total of 5 μL of fMLP solution or vehicle control was injected into the right hindlimb knee joint using a 5 μL Hamilton microliter syringe (Hamilton, 7634-01) and 27-gauge needle. The dosing of 5 μg of fMLP was chosen based on its maximum solubility in vehicle solution and maximum volume of solution that could be injected into the knee joint space. The syringe needle was applied to the intra-articular space directly through the patellar ligament with the knee flexed at a 90° angle with needle administration perpendicular to the apex of the flexed knee.

### Implant Surgery

Briefly, 2 days prior to surgery, the ventral and lateral surface of the right hindlimb of 12-week-old C57BL/6J mice were shaved. To ensure additional removal of hair, Nair hair removal spray was used. The day prior to surgery, all non-sterilized instruments and implant material were autoclaved in self-sealing autoclave pouches. On the day of surgery, mice were first anesthetized with 2% isoflurane gas anesthesia or an injection mixture of ketamine (90 mg/kg) and xylazine (4.5 mg/kg). The right leg was scrubbed with 70% ethanol followed by iodine. Using aseptic technique and under guidance of a dissection microscope (Zeiss, Stemi 508), a skin incision over the right knee was performed followed by a medial parapatellar arthrotomy. Incisions were performed with a Micro Knives sterile scalpel (Fine Science Tools, 10315-12). To expose the femoral condyles, lateral displacement of the quadriceps patellar complex was performed. The intercondylar notch was located; a 25-gauge syringe needle attached to a 3-mL syringe was used to penetrate the intercondylar notch and ream the distal intramedullary canal at a distance of approximately 10 mm. An orthopedic-grade stainless steel Kirschner wire (K-wire) (diameter 0.6 mm; Depuy Synthes) was surgically placed in a retrograde fashion into intramedullary canal with assistance of a pin holder (Fine Sciences Tools, 26018-17). The distal aspect of the K-wire was cut to a length of approximately 11 mm with a wire cutter leaving approximately 1 mm protruding into the joint space.

### Bioluminescence Imaging (BLI)

IVIS® Lumina II In Vivo Imaging System (Perkin Elmer) was used to track neutrophil activity at the knee joint. For the assessment of neutrophil response in mice receiving knee injection of fMLP or vehicle control, BLI was performed at baseline prior to injection or surgery, as well as at 2 hours and 6 hours post injection or surgery. At 8 minutes prior to each imaging time-point, mice received intra-peritoneal (I.P.) injection of 100 mg/kg of luminol (Sigma-Aldrich, A4685) in PBS solution. Luminol is a chemiluminescent compound that produces a bioluminescent signal in the presence of reactive oxygen species (ROS) catalyzed by myeloperoxidase (MPO) in neutrophils.

The above Figures and disclosure are intended to be illustrative and not exhaustive. This description will suggest many variations and alternatives to one of ordinary skill in the art. All such variations and alternatives are intended to be encompassed within the scope of the attached claims. Those familiar with the art may recognize other equivalents to the specific embodiments described herein which equivalents are also intended to be encompassed by the attached claims.

### REFERENCES

1. Ruan Q, Yang K, Wang W, Jiang L, Song J. Clinical predictors of mortality due to COVID-19 based on an analysis of data of 150 patients from Wuhan, China. Intensive Care Med 2020; 46(5):846-848. DOI: 10.1007/s00134-020-05991-x.
2. Wu C, Chen X, Cai Y, et al. Risk Factors Associated With Acute Respiratory Distress Syndrome and Death in Patients With Coronavirus Disease 2019 Pneumonia in Wuhan, China. JAMA Intern Med 2020. DOI: 10.1001/jamainternmed.2020.0994.
3. Herrera-Garcia A M, Dominguez-Luis M J, Arce-Franco M, et al. Prevention of neutrophil extravasation by alpha2-adrenoceptor-mediated endothelial stabilization. J Immunol 2014; 193(6):3023-35. DOI: 10.4049/jimmunol.1400255.
4. Flanders C A, Rocke A S, Edwardson S A, Baillie J K, Walsh T S. The effect of dexmedetomidine and clonidine on the inflammatory response in critical illness: a systematic review of animal and human studies. Crit Care 2019; 23(1):402. DOI: 10.1186/s13054-019-2690-4.
5. Pearson S D, Patel B K. Evolving targets for sedation during mechanical ventilation. Curr Opin Crit Care 2020; 26(1):47-52. DOI: 10.1097/MCC.0000000000000687.
6. Quan H, Li B, Couris C M, et al. Updating and validating the Charlson comorbidity index and score for risk adjustment in hospital discharge abstracts using data from 6 countries. Am J Epidemiol 2011; 173(6):676-82. DOI: 10.1093/aje/kwq433.
7. Group R C, Horby P, Lim W S, et al. Dexamethasone in Hospitalized Patients with Covid-19—Preliminary Report. The New England journal of medicine 2020. DOI: 10.1056/NEJMoa2021436.
8. Sterne J A C, Murthy S, Diaz J V, et al. Association Between Administration of Systemic Corticosteroids and Mortality Among Critically III Patients With COVID-19: A Meta-analysis. JAMA 2020 (In eng). DOI: 10.1001/jama.2020.17023.
9. Thompson B T, Chambers R C, Liu K D. Acute Respiratory Distress Syndrome. N Engl J Med 2017; 377(19):1904-1905. DOI: 10.1056/NEJMc1711824.
10. Barnes B J, Adrover J M, Baxter-Stoltzfus A, et al. Targeting potential drivers of COVID-19: Neutrophil extracellular traps. J Exp Med 2020; 217(6). DOI: 10.1084/jem.20200652.
11. Laforge M, Elbim C, Frère C, et al. Tissue damage from neutrophil-induced oxidative stress in COVID-19. Nat Rev Immunol 2020; 20(9):515-516. (In eng). DOI: 10.1038/s41577-020-0407-1.
12. Schurink B, Roos E, Radonic T, et al. Viral presence and immunopathology in patients with lethal COVID-19: a prospective autopsy cohort study. Lancet Microbe 2020 (In eng). DOI: 10.1016/S2666-5247(20)30144-0.
13. Li K, Hao Z, Zhao X, Du J, Zhou Y. SARS-CoV-2 infection-induced immune responses: Friends or foes? Scand J Immunol. 2020; 92(2):e12895. Epub 2020 May 24. doi: 10.1111/sji.12895.
14. Land W G. Role of DAMPs in respiratory virus-induced acute respiratory distress syndrome-with a preliminary reference to SARS-CoV-2 pneumonia. Genes Immun. 2021; 22(3):141-60. Epub 2021 Jun. 19. doi: 10.1038/s41435-021-00140-w.

1. A method of treating a viral respiratory syndrome in a subject, the method comprising:
administering a non-sedative therapeutically effective amount of an α-2 adrenergic receptor (AR) agonist to the subject.

2. The method according to claim 1, wherein the α-2 AR agonist is selected from the group consisting of Dexmedetomidine, Clonidine, Guanfacine, Guanabenz, Guanoxabenz, Guanethidine, Xylazine, Tizanidine, Medetomidine, Methyldopa, Methylnorepinephrine, Fadolmidine, Iodoclonidine, Apraclonidine, Detomidine, Lofexidine, Amitraz, Mivazerol, Azepexol, Talipexol, Rilmenidine, Naphazoline, Oxymetazoline, Xylometazoline, Tetrahydrozoline, Tramazoline, Talipexole, Romifidine, propylhexedrine, Norfenefrine, Octopamine, Moxonidine, Lidamidine, Tolonidine, UK14304, DJ-7141, ST-91, RWJ-52353, TCG-1000, 4-(3-aminomethyl-cyclohex-3-enylmethyl)-1,3-dihydro-imidazole-2-thione, and 4-(3-hydroxymethyl-cyclohex-3-enylmethyl)-1,3-dihydro-imidazole-2-thione or a pharmaceutically acceptable salt thereof.

3. The method according to claim 1, wherein the α-2 AR agonist is Dexmedetomidine or Clonidine.

4. The method according to claim 1, wherein the administration of the α-2 AR agonist is oral, sublingual, intranasal or transdermal.

5. The method according to claim 1, wherein the viral respiratory syndrome is caused by a virus selected from the group consisting of Severe Acute Respiratory Syndrome Coronavirus 2 (SARS-CoV-2), rhinoviruses, influenza (Flu A and B), adenoviruses, picornaviruses, respiratory syncytial virus, parainfluenza viruses 1-3, human metapneumovirus, coronaviruses, Middle East Respiratory Syndrome Coronavirus (MERS-CoV), Severe Acute Respiratory Syndrome Coronavirus (SARS-CoV)) and herpesviruses.

6. The method according to claim 1, wherein the viral respiratory syndrome is caused by SARS-CoV-2.

7. The method according to claim 1, wherein the α-2 AR agonist is administered prior to use of a ventilator.

8. The method according to claim 1, wherein the subject is an outpatient.

9. A method of treating a viral respiratory syndrome in a subject, the method comprising:
administering a therapeutically effective amount of an α-2 adrenergic receptor (AR) agonist and a therapeutically effective amount of an α-1 adrenergic receptor (AR) antagonist to the subject.

10. The method of claim 9, wherein the α2 adrenergic receptor agonist is delivered in a non-sedative dose.

11. The method according to claim 9, wherein the α-2 AR agonist is selected from the group consisting of Dexmedetomidine, Clonidine, Guanfacine, Guanabenz, Guanoxabenz, Guanethidine, Xylazine, Tizanidine, Medetomidine, Methyldopa, Methylnorepinephrine, Fadolmidine, Iodoclonidine, Apraclonidine, Detomidine, Lofexidine, Amitraz, Mivazerol, Azepexol, Talipexol, Rilmenidine, Naphazoline, Oxymetazoline, Xylometazoline, Tetrahydrozoline, Tramazoline, Talipexole, Romifidine, propylhexedrine, Norfenefrine, Octopamine, Moxonidine, Lidamidine, Tolonidine, UK14304, DJ-7141, ST-91, RWJ-52353, TCG-1000, 4-(3-aminomethyl-cyclohex-3-enylmethyl)-1,3-dihydro-imidazole-2-thione, and 4-(3-hydroxymethyl-cyclohex-3-enylmethyl)-1,3-dihydro-imidazole-2-thione or a pharmaceutically acceptable salt thereof.

12. The method according to claim 9, wherein the α-2 AR agonist is Dexmedetomidine or Clonidine.

13. The method according to claim 9, wherein the α-1 AR antagonist is selected from the group consisting of alfuzosin, dihydroergotamine mesylate, doxazosin, ergotamine, phentolamine mesylate, phenoxybenzamine, prazosin, silodosin, tamsulosin, terazosin, and tolazoline

14. The method according to claim 10, wherein the administration of the α-2 AR agonist is oral, sublingual, intranasal or transdermal.

15. The method according to claim 9, wherein the viral respiratory syndrome is caused by a virus selected from the group consisting of Severe Acute Respiratory Syndrome Coronavirus 2 (SARS-CoV-2), rhinoviruses, influenza (Flu A and B), adenoviruses, picornaviruses, respiratory syncytial virus, parainfluenza viruses 1-3, human metapneumovirus, coronaviruses, Middle East Respiratory Syndrome Coronavirus (MERS-CoV), Severe Acute Respiratory Syndrome Coronavirus (SARS-CoV)) and herpesviruses.

16. The method according to claim 9, wherein the viral respiratory syndrome is caused by SARS-CoV-2.

17. The method according to claim 10, wherein the α-2 AR agonist is administered prior to use of a ventilator.

18. The method according to claim 10, wherein the subject is an outpatient.
